(19) United States
(12) Patent Application Publication
(10) Pub. No.: US 2016/0030764 A1
(43) Pub. Date: Feb. 4, 2016

(54) NON-TACTILE SENSORY SUBSTITUTION DEVICE

(71) Applicant: Allan C. ENTIS, Tel Aviv (IL)

(72) Inventor: Allan C. Entis, Tel Aviv (IL)

(21) Appl. No.: 14/774,140

(22) PCT Filed: Mar. 16, 2014
(86) PCT No.: PCT/IL2014/050292

§ 371 Date: Sep. 10, 2015

Related U.S. Application Data
(60) Provisional application No. 61/787,435, filed on Mar. 15, 2013.

(51) Int. Cl. A61N 5/06; A61N 7/00

(52) U.S. Cl. CPC A61N 5/0622; A61N 7/00; A61N 2007/0026

(57) ABSTRACT

There is therefore provided in accordance with an embodiment of the invention, apparatus for providing a person with neural signals responsive to features of an environment, the apparatus comprising: an environment imager that acquires an image of a region of the person's environment; and a non-contact corneal neural stimulator that stimulates nerve endings in the cornea of the person's eye responsive to the image of the environment.

### RELATED APPLICATIONS

The present application claims the benefit under 35 U.S.C. 119(e) of U.S. Provisional Application 61/787,435, filed on Mar. 15, 2013, the disclosure of which is incorporated herein by reference.

### TECHNICAL FIELD

Embodiments of the invention relate to sensory substitution devices (SSDs), in which non-retinal stimulus is used to generate input to the brain of visually compromised people to substitute for damage or loss of retinal input.

### BACKGROUND

A person's perception of the environment responsive to sensory signals received from a sense organ such as an eye, an ear, or the skin, appears to be a subjective impression that the person's brain constructs to organize and correlate the sensory signals and provide the person with a conscious model that operates to interface the person with the environment. The interface enables the person to observe, record, and direct, his or her responses to features of the environment that are represented by the sensory signals.

The way the brain uses sensory signals from a given sense organ to generate a perception, and an efficiency with which it does so, appears at least partially as functions of learning, and different parts of the brain exhibit substantial plasticity in learning to generate perceptions responsive to signals from different sensory organs.

For example, the occipital cortex of the brain in healthy people is responsible for providing visual perceptions and spatial models of an environment responsive to light collected from the environment by the eyes. In blind people who have suffered or been born with damage to the eyes or the neural system that transports signals from the eyes to the occipital cortex, the occipital cortex does not receive signals from the eyes. However, functional magnetic resonance imaging (fMRI) shows that the occipital cortex in blind people often adapts itself to process audio signals generated by the ears and tactile signals generated by the fingers. For example, the occipital cortex is generally involved in processing tactile signals produced by the fingers of a blind person when he or she touches Braille letters to provide the person with perceptions of the letters.

Blind people also appear to use the occipital cortex to generate spatial models of environments responsive to tactile or audio signals that are reminiscent of spatial models supported by visual perceptions in people who have normal sight. The spatial models that the blind appear to generate responsive to non retinal signaling enable them to function and navigate their environments in a manner that implies that their models share traits that characterize the models of sighted people.

The plasticity of the brain in learning to process sensory signals has been demonstrated in the development and use of SSDs that are designed to provide blind people with audio or tactile sensory input that substitutes for retinal signaling that they do not have. For example, in an SSD technology referred to as “vOICe”, images of an environment acquired by a video camera mounted in a pair of glasses worn by a blind person are encoded in auditory signals. The auditory signals are provided to the person by stereo speakers mounted in the glasses to aid the person in interacting with, and navigating in, the environment. Blind users of the glasses have reported and demonstrated that the audio signals they receive from the SSD enable them to distinguish visual features, such as objects and patterns, of the environment.

Some tactile SSD technologies convert images from a glasses mounted camera to electrical signals on small electrodes arrayed in a tongue display unit (TDU) worn on the tongue. The electrical signals generated responsive to an image acquired by the camera stimulate tactile sensations on small regions of the tongue to generate an image, a “tongue image”, on the tongue that represents the camera image. BrainPort® technologies of WICO Inc in Wisconsin USA reports developing a 3 centimeter (cm)×3 cm TDU having about 600 electrodes for generating tongue images. Blind people using the device appear to perceive the tongue images as low resolution images of their environment and are able to use the tongue images to distinguish such features as another person's fingers or to play tic-tac-toe on a large (about 30 cm on a side) tic-tac-toe grid.

### SUMMARY

An aspect of an embodiment of the invention relates to providing a high resolution non-tactile SSD (NT-SSD) that provides non-tactile stimulation of nerve endings, optionally in regions of the cornea, to generate neural signals that substitute for retinal signals that may be non-existent or impaired in a vision compromised person. The NT-SSD stimulates nerve endings in a given region of, optionally, the cornea without involving contact of the given region by a material body to generate the neural signals by radiating and focusing radiated energy onto the region.

In an embodiment of the invention, the radiated energy comprises electromagnetic energy that may generate a temperature change and/or a photoacoustic wave in the given corneal region. In an embodiment of the invention, the radiated energy comprises acoustic energy that may generate a change in pressure or temperature in the corneal region.

The neural signals, also referred to as non-tactile corneal stimulation (NTCS) neural signals, that are generated by the radiated energy encode and provide the person's brain with a representation of an image of an environment in which the vision compromised person is present that would normally be represented by retinal signals produced by the retina of a person having healthy vision. Hereinafter, the representation provided by the NTCS neural signals is also referred to as an “NTCS image”.

In an embodiment of the invention, an NT-SSD comprises an environment imager, such as a camera, that acquires an image of an environment in which a person, who may be blind or vision compromised, is present, a gaze tracker, and an energy radiation apparatus (ERA), which may also be referred to as a “non-contact neural stimulator”. The gaze tracker tracks a location and/or direction of gaze of an eye of the blind person. The ERA radiates and focuses energy that represents the camera image to relatively small localized regions of the cornea in a pattern that is registered to the pupil location and/or gaze direction determined by the gaze tracker. The energy focused to a given small localized region of the cornea stimulates nerve endings in the region. The stimulated nerve endings generate NTCS neural signals responsive to the stimuli. The pattern of NTCS neural signals encodes the camera image mapped onto the cornea by the focused energy into a NTCS image. The NTCS neural signals, and thereby the NTCS image, propagate to the brain along sensory nerve fibers to which the nerve endings are coupled. Following practice and training using the NT-SSD, the person's brain is expected to use NTCS neural images it receives to generate representations of environments imaged by the camera that enable the blind person to distinguish features of the environments.

An aspect of an embodiment of the invention relates to providing a holistic method of training a blind person to use an NT-SSD or a SSD. In the holistic training method, an object imaged by an NT-SSD or an SSD being used by a blind person is moved in a field of vision (FOV) of the NT-SSD or SSD and its movement is accompanied by a non-visual stimulus or stimuli, which correspond to the location and movement of the object.

A non-visual stimulus used in a holistic training method in accordance with an embodiment of the invention may be sound associated with the object, and location of the object may be sensed by the blind person's auditory system. Optionally, the sound is changed corresponding to the movement. For example, as the object is moved towards or away from the blind person, the motion may be accompanied by a sound that respectively increases and decreases in pitch. Optionally, the non-visual stimulus comprises an odor that the object emits and may be sensed by the blind person. As the object moves in the FOV of the NT-SSD or SSD, a location of the object may be sensed as a location perceived by the blind person's olfactory senses as a location of a source of the odor. The addition of non-visual stimuli that correspond to motion and location of the object aids the blind person in learning to use NTCS neural images, or SSD images, to provide images of an environment that aid the person in interfacing and functioning in the environment.

Whereas in the above description a NT-SSD is described as stimulating a region of the cornea, practice of the invention is not limited to non-tactile stimulation of the cornea. An NT-SSD may be configured to focus energy to regions of the sclera to generate neural signals that may be used by a vision impaired person to generate an internal mental image of his or her environment. Hereinafter, non-tactile neural signals and corresponding images stimulated by an NT-SSD in a region of the cornea or a region of the sclera of a person's eye are generically referred to as NTCS neural signals and images.

It is further noted that whereas an NT-SSD is described as being used as a substitute or aid to vision by a vision compromised person, an NT-SSD may be used to provide a person, whether vision compromised or healthy, with data, which is encoded in a pattern of non-tactile neural stimuli of the eye generated by the NT-SSD. For example, a camera in an NT-SSD may image a visually healthy person's (or a vision impaired person) surroundings with light from the non-visual spectrum. The light may be infrared (IR) light and the NT-SSD may stimulate the person's cornea and/or sclera responsive to the IR image to generate an IR image on the person's eye that “overlays” what the person sees with visual light and provides the person with an additional layer of data to characterize his or her perception of the environment.

In an embodiment of the invention, the NT-SSD may image an environment with acoustic waves and stimulate a person's cornea with signals responsive to an acoustic image of the environment. The acoustic waves may for example be radiated at frequencies used by bats and NTCS neural signals and images generated responsive to the stimulation may provide the person with a sense or image of the person's environment. The sense or image that the person generates responsive to the “acoustic” NTCS image may exhibit features that have cognates with how a bat sees an environment.

There is therefore provided in accordance with an embodiment of the invention, an apparatus for providing a person with neural signals responsive to features of an environment, the apparatus comprising: an environment imager that acquires an image of a region of the person's environment; and a non-contact corneal neural stimulator tactile that stimulates nerve endings in the cornea of the person's eye responsive to the image of the environment.

In an embodiment of the invention, the environment imager comprises a camera. Optionally, the camera images the environment with light in the visible spectrum. Additionally or alternatively the camera images the environment with light from the non-visible spectrum. Optionally, the light from the non-visible spectrum comprises infrared (IR) light.

In an embodiment of the invention the environment imager comprises an acoustic imager that images the environment with acoustic waves. Optionally, the acoustic imager radiates the acoustic waves at acoustic frequencies used by bats to image the environment.

In an embodiment of the invention, the non-contact corneal neural stimulator comprises a source of electromagnetic waves that focuses electromagnetic energy onto a plurality of different regions of the cornea to stimulate the nerve endings. Optionally, the electromagnetic energy generates a temperature change in a region of the different regions to which the electromagnetic energy is focused. Additionally or alternatively, the electromagnetic energy may generate a photoacoustic wave in a region of the different regions to which the electromagnetic energy is focused.

In an embodiment of the invention, the non-contact corneal neural stimulator comprises a source of acoustic waves that focuses acoustic energy onto a plurality of different regions of the cornea to stimulate the nerve endings. Optionally, the acoustic energy generates a temperature change in a region of the different regions to which the acoustic energy is focused. Additionally or alternatively, the acoustic energy optionally generates a pressure change in a region of the different regions to which the acoustic energy is focused.

In an embodiment of the invention the apparatus comprises a gaze tracker that determines a gaze direction of the person's eye. Optionally, the neural stimulator stimulates a region of the cornea responsive to a location of the region referenced with respect to the gaze direction. Optionally, the reference with respect to the gaze direction comprises a reference with respect to the pupil. Additionally or alternatively, the reference with respect to the gaze direction may comprise a reference with respect to a fixed feature of the eye.

In an embodiment of the invention the neural stimulator stimulates the nerve endings to generate a pattern of stimulated nerve endings that encodes the image.

In an embodiment of the invention the neural stimulator stimulates the nerve endings to provide the person with data.

There is also provided in accordance with an embodiment of the invention, an apparatus for providing a person with data, the apparatus comprising a non-contact corneal neural stimulator that focuses radiated energy onto a plurality of different regions of the cornea to stimulate the nerve endings in a pattern that encodes the data.

There is further provided in accordance with an embodiment of the invention, a method of inputting data to a person, the method comprising: acquiring an image of the person's environment; and focusing radiated energy onto a plurality of different regions of the cornea to stimulate the nerve endings in a pattern that encodes the data. Optionally, the radiated energy comprises electromagnetic energy. Additionally or alternatively, the radiated energy may comprise acoustic energy. In an embodiment of the invention, the data represents an image of the person's environment. In an embodiment of the invention, the data comprises acquiring the image. Acquiring the image optionally comprises using a camera to acquire the image. Acquiring the image may comprise using an acoustic imager to acquire the image.

In the discussion, unless otherwise stated, adverbs such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one, or any combination of more than one of items it conjoins.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF FIGURES

Non-limiting examples of embodiments of the invention are described below with reference to figures attached hereto that are listed following this paragraph. Identical structures, elements or parts that appear in more than one figure are generally labeled with a same numeral in all the figures in which they appear. Dimensions of components and features shown in the figures are chosen for convenience and clarity of presentation and are not necessarily shown to scale.

FIG. 1 schematically shows a non-tactile sensory substitution device (NT-SSD) comprising a gaze tracker and laser stimulators for generating non-tactile corneal stimulation NTCS neural signals, in accordance with an embodiment of the invention; and

FIG. 2 schematically shows an NT-SSD comprising acoustic stimulators for generating NTCS neural signals, in accordance with an embodiment of the invention.

### DETAILED DESCRIPTION

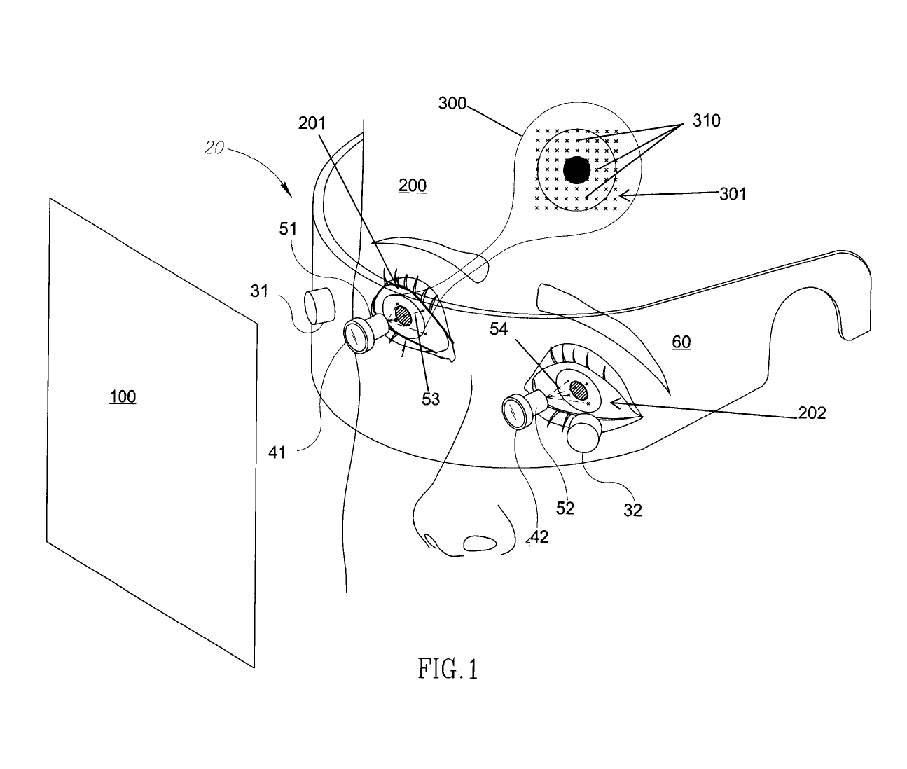

FIG. 1 schematically shows an NT-SSD 20 providing non-tactile corneal stimulation optionally to a vision compromised person 200 responsive to an environment represented by a rectangle 100, in accordance with an embodiment of the invention.

NT-SSD 20 optionally comprises right eye and left eye cameras 41 and 42 respectively, right eye and left eye laser stimulators 51 and 52 respectively, and right eye and left eye gaze trackers 31 and 32 respectively, optionally mounted to a visor 60. Right and left eye cameras 41 and 42 optionally acquire right and left images of environment 100 substantially from the perspectives of right and left eyes 201 and 202 of person 200. Right and left laser stimulators 51 and 52 receive data from cameras 41 and 42 associated with the right and left images and direct laser beams 53 and 54 respectively to illuminate and stimulate a raster of regions on the corneas, and optionally sclera, of eyes 201 and 202 to generate NTCS neural images responsive to the received data that may provide a sense of depth or perspective. By way of example, an inset 300 shows an enlarged portion of the cornea and portions of the sclera of right eye 201 of person 200 and a raster 301 of regions 310 of the cornea and sclera that are stimulated by laser beam 51.

In an embodiment of the invention locations of stimulated regions 310 of the cornea and/or sclera are registered to a location of the person's pupil and/or gaze direction and or location of a fixed feature on the eyeball surface provided by gaze tracker 31. Unless otherwise indicated or obvious from the context, referencing a location of a stimulated region to a gaze direction includes referencing the location to the pupil, gaze direction, or fixed feature. Referencing a location of a stimulated region to a gaze direction may, as discussed by way of example below, be advantageous for stimulating regions of the cornea appropriate to provide person 200 with an NTCS image that may satisfactorily track features in environment 100 as the person moves, changes gaze direction or the features move.

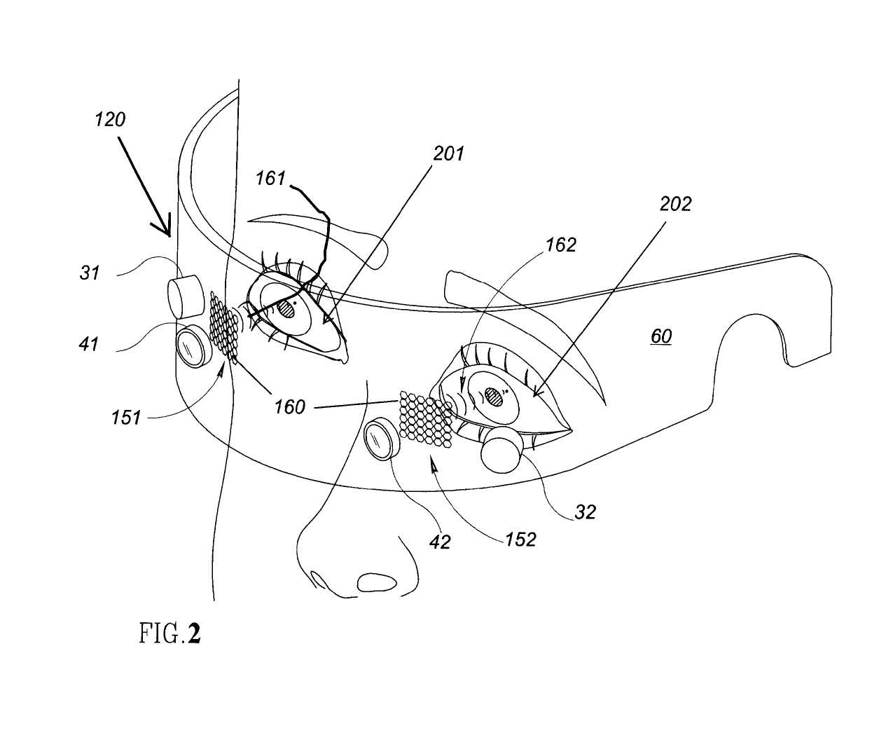

Gaze trackers 31 and 32 may determine gaze direction using any of various methods known in the art, and may for example determine gaze direction responsive to imaging eye glints and/or Purkinje reflections or by determining a direction of a magnetic dipole field that the eye generates. In referencing locations for stimulation, laser stimulator 51 or 52 or gaze tracker 31 or 32 may use a reference coordinate system, such as a coordinate system having x and y axes, that the laser stimulator or its associated gaze tracker establishes. Optionally the reference coordinate system has x and y axes and laser stimulator 51 or 52 may stimulate the eye as a function of x and y coordinates referenced to the coordinate system. Optionally, an origin of the coordinate system is the center of the pupil. Optionally, the coordinate system has x and y axes that are substantially fixed relative to the structure of the eye and do not move relative to the surface of the eye with movement of the eye in its socket. Fixed x and y axes may be established responsive to a fixed feature of the eye, such as a mark on the eye or an asymmetry of the eye. A fixed coordinate system may be a virtual fixed coordinate system having axes whose directions are determined from physiological knowledge as to how the musculature that controls motion of an eye in its socket would move fixed features of the eye as the musculature moves the eye.

Whereas in FIG. 1 NT-SSD 20 comprises laser stimulators 51 and 52, practice of the invention is not limited to using laser stimulators to generate NTCS neural signals. For example, FIG. 2 schematically shows an NT-SSD 120 comprising right and left acoustic stimulators 151 and 152 comprising phased arrays 160 for generating focused acoustic beams 161 and 162 to stimulate regions of the right and left eyes 201 and 202 respectively, of person 200 to generate NTCS neural signals and images.

It also noted that whereas environment imagers in FIGS. 1 and 2 are indicated as comprising cameras 41 and 42, which acquire optical images of the environment of person 200, in accordance with an embodiment of the invention environment imagers that comprise acoustic imagers may be used to acquire images of a person's environment to provide data for generating NTCS neural signals. And whereas NT-SSD 20 and 120 comprise right and left environment imagers 41 and 42 and stimulators 51, 52 and 151, 152 respectively and are indicated as possibly providing perspective NTCS neural images, an embodiment of the invention may of course have only one environment imager and optionally only one stimulator. It is further noted that the placement of environment imagers and stimulators in FIGS. 1 and 2 may of course be different from placement of the imagers and stimulators shown in FIGS. 1 and 2. In the figures, the imagers and/or stimulators may block portions of the field of view (FOV) of person 200. Whereas for a blind person blockage of the field of view may be irrelevant, for a sighted person using an NT-SSD for receiving data in accordance with an embodiment of the invention, blockage of the person's field of view may be undesirable. For such cases, environment imagers may be located outside of the person's FOV or at a periphery of the FOV. For example, environment imagers may be located on sides of visor 60 and stimulators may be mounted off-center, or substantially off center from a central region of the person's FOV when the person is looking straight ahead relative to head orientation. Furthermore, whereas components of NT-SSD 20 and 120 are shown mounted to a visor, components of an NT-SSD are not necessarily mounted on a visor or directed to image an environment in front of person 200. For example, cameras 41 and 42 may be mounted on a person's chest or may be mounted to the back of a person's head or the person's back to provide images of an environment behind the person.

In the description and claims of the present application, each of the verbs, “comprise” “include” and “have”, and conjugates thereof, are used to indicate that the object or objects of the verb are not necessarily a complete listing of components, elements or parts of the subject or subjects of the verb.

Descriptions of embodiments of the invention in the present application are provided by way of example and are not intended to limit the scope of the invention. The described embodiments comprise different features, not all of which are required in all embodiments of the invention. Some embodiments utilize only some of the features or possible combinations of the features. Variations of embodiments of the invention that are described, and embodiments of the invention comprising different combinations of features noted in the described embodiments, will occur to persons of the art. The scope of the invention is limited only by the claims.

1. Apparatus for providing a person with neural signals responsive to features of an environment, the apparatus comprising:
an environment imager that acquires an image of a region of the person's environment; and
a non-contact corneal neural stimulator that stimulates nerve endings in the cornea of the person's eye responsive to the image of the environment.

2. The apparatus according to claim 1 wherein the environment imager comprises a camera.

3. The apparatus according to claim 2 wherein the camera images the environment with light in the visible spectrum.

4. The apparatus according to claim 2 wherein the camera images the environment with light from the non-visible spectrum.

5. The apparatus according to claim 4 wherein the light from the non-visible spectrum comprises infrared (IR) light

6. The apparatus according to claim 1 wherein the environment imager comprises an acoustic imager that images the environment with acoustic waves.

7. The apparatus according to claim 6 wherein the acoustic imager radiates the acoustic waves at acoustic frequencies used by bats to image the environment.

8. The apparatus according to claim 1 wherein the non-contact corneal neural stimulator comprises a source of electromagnetic waves that focuses electromagnetic energy onto a plurality of different regions of the cornea to stimulate the nerve endings.

9. The apparatus according to claim 8 wherein the electromagnetic energy generates a temperature change in a region of the different regions to which the electromagnetic energy is focused.

10. The apparatus according to claim 8 wherein the electromagnetic energy generates a photoacoustic wave in a region of the different regions to which the electromagnetic energy is focused.

11. The apparatus according to claim 1 wherein the non-contact corneal neural stimulator comprises a source of acoustic waves that focuses acoustic energy onto a plurality of different regions of the cornea to stimulate the nerve endings.

12. The apparatus according to claim 11 wherein the acoustic energy generates a temperature change in a region of the different regions to which the acoustic energy is focused

13. The apparatus according to claim 11 wherein the acoustic energy generates a pressure change in a region of the different regions to which the acoustic energy is focused

14. The apparatus according to claim 1 and comprising a gaze tracker that determines a gaze direction of the person's eye.

15. The apparatus according to claim 14 wherein the neural stimulator stimulates a region of the cornea responsive to a location of the region referenced with respect to the gaze direction.

16. The apparatus according to claim 15 wherein the reference with respect to the gaze direction comprises a reference with respect to the pupil.

17. The apparatus according to claim 15 wherein the reference with respect to the gaze direction comprises a reference with respect to a fixed feature of the eye.

18. The apparatus according to claim 1 wherein the neural stimulator stimulates the nerve endings to generate a pattern of stimulated nerve endings that encodes the image.

19. The apparatus according to claim 1 wherein the neural stimulator stimulates the nerve endings to provide the person with data.

20. Apparatus for providing a person with data, the apparatus comprising a non-contact corneal neural stimulator that focuses radiated energy onto a plurality of different regions of the cornea to stimulate the nerve endings in a pattern that encodes the data.

21-27. (canceled)
